SEQUENCE LISTING (22 sequences)

SEQ ID NO: 1 - DNA, 2040 bp, Neisseria meningitidis
  CDS (1)..(2040)
atgatgtcgcccgaaacccgaaaacagctcaaaatcaccgatgtttccgccaagaagctcgacaaactcaacctccataccgcgtgggatttggtgttgcacctgccgctgcgttacgaggacgagacgcacattatgccgattaaggacgcgccgattggcgtgccgtgtcaggtcgagggggaggttatccatcaggaagtaacgttcaaaccgcgcaagcagctgattgtccaaatcgccgacggctccggcagcgtcctttttctgcgcttcatccacttttacgccagccatcagaaacagacggcgatcggcaaacgcatccgcgccgtgggcgaaatcaaacacggcttttacggcgacgagatgattcatcccaaaatccgcgatgccgagggcggcggtttggcggaaagcctcacgccggtttacccgaccgtaaacggtttgaaccagcccactttgcgccgtattattcagacggcgttggacgttacgccgctgcacgacacgctgcccgatgccctgctgtgccgtctgaagctgccacacctcgccgaaagcctgcgtcttttgcattcgccgccgccgagtttcacgattcatcagctttcagacggcacgctgcctgcatggcaacggctcaaattcgacgaacttttggcgcaacagctttcgatgcgcttggcgcgacagaagcgtatcggcggcacggcggcggcattgggcggcgacggcacattgacccaagccctgcgccaagccctgccgtttgccctgaccgatgcgcaagaaaaagttgtttccgaaatctgccgcgatatggcgcaaacctacccgatgcaccgcctgctgcagggcgatgtcggcagcggcaaaaccattgtggctgctttgtctgctttgacggctatcgaatccggtgcgcaggtggctgtaatggcgcccactgaaatccttgccgaacagcattttattaaatttaaacaatggctcgaacctttgggcattgaagttgtccgcctttttggcagtttgcgtaaaaaagccaaagacgaagccaaagccaaactggctgacggcagcgtcaaaatcgccgtcggcacgcacgccctgttttcagacggcgtggcgtttcacaatttgggcttgaccattgtggacgaacagcaccgtttcggcgttgcccaacgcctcgccctcaaaaacaaagggcgcgaagtccatcagctgatgatgtccgccacgcccatcccgcgcacgcttgcgatgagttttttcgccgatttggacgtgtccgtcatcgacgaattgccgcccgggcgcacaccgattaaaacgcgcctcgtcaacaacgtccgccgcgccgaagtcgaaggcttcgtcctcggcacttgccgaaaagggcggcaggcgtattgggtctgcccattgattgaagaaagcgaaaccctgcaactgcaaaccgccgccgaaaccctcgcccggcttcagacggcattgcccgaactcaatatcggactggtacacgggcgcatgaaggccgccgaaaaggccgaagtgatggcgcggttttcttcgggggggctgaacgtcttggtcgccaccaccgtcatcgaagtcggcgtagatgtgcccaatgccgccctgatggtcatcgaacacgccgagcgcatgggcttggcgcagcttcaccaattacgcggacgggtagggcgcggcgcggcggaaagcgtgtgcgtcctcctgtttgccgaacccttgggcgaactcgccaaagcgcggctgaaagtcatctacgaacacaccgacggcttcgaaatcgcccgccaagacctcaatatccgcggcccgggcgagtttctcggcgcgcgccaaagcggcgtgcctatgctgcgtttcgccaaactcgaagaagacctgcaccttttggaacaggcgcgcgaaaccgccccgatgctgattgagcaaaaccccgaaatcgtcgaagcgcatttggcaaggtggctttccggcagggaagggtatttgggcgtg

SEQ ID NO: 2 - protein, 680 aa, Neisseria meningitidis
MMSPETRKQLKITDVSAKKLDKLNLHTAWDLVLHLPLRYEDETHIMPIKDAPIGVPCQVEGEVIHQEVTFKPRKQLIVQIADGSGSVLFLRFIHFYASHQKQTAIGKRIRAVGEIKHGFYGDEMIHPKIRDAEGGGLAESLTPVYPTVNGLNQPTLRRIIQTALDVTPLHDTLPDALLCRLKLPHLAESLRLLHSPPPSFTIHQLSDGTLPAWQRLKFDELLAQQLSMRLARQKRIGGTAAALGGDGTLTQALRQALPFALTDAQEKVVSEICRDMAQTYPMHRLLQGDVGSGKTIVAALSALTAIESGAQVAVMAPTEILAEQHFIKFKQWLEPLGIEVVRLFGSLRKKAKDEAKAKLADGSVKIAVGTHALFSDGVAFHNLGLTIVDEQHRFGVAQRLALKNKGREVHQLMMSATPIPRTLAMSFFADLDVSVIDELPPGRTPIKTRLVNNVRRAEVEGFVLGTCRKGRQAYWVCPLIEESETLQLQTAAETLARLQTALPELNIGLVHGRMKAAEKAEVMARFSSGGLNVLVATTVIEVGVDVPNAALMVIEHAERMGLAQLHQLRGRVGRGAAESVCVLLFAEPLGELAKARLKVIYEHTDGFEIARQDLNIRGPGEFLGARQSGVPMLRFAKLEEDLHLLEQARETAPMLIEQNPEIVEAHLARWLSGREGYLGV

SEQ ID NO: 3 - DNA, 1257 bp, Neisseria meningitidis
  CDS (1)..(1257)
atgcacgtctccgaattacaaaccctgcacatttccaaactcttagaattggcggaagaacacggcatcgaaaacgccaaccgattccgcaaacaagatctcgtatttgccatcgtccgccagatgatgaaaaaaggcgagggtttcacctgctccggcacgcttgaaatcctgcccgacggcttcggcttcctccgcagcgcggacacgtcctatcttgccggccccgacgacatctatgtctcgcccacccaaatccgccgcttcaacctgcatacgggcgacaccatcgaaggaagcgtgcgcgtcccaaaagacaacgaacgctattttgccctggtcaggcttgataccatcaacggcgaccacccggaagtatgccgccataaaatcctgtttgaaaacctgaccccgctgtttccgaccgaacagttgaagctggaacgcgacttaaagtccgaagaaaacctgaccggacgtgccatcgacctgatttcccctatcggcaaaggtcagcgcgccctcttggttgccccgcccaaaagcggtaaaaccgtgatgctgcaaaacattgcccacgccgttacctcaaactatcccgaagtcgaactcatcgtcctcttgattgacgagcgtcccgaagaagtaaccgaaatgagccgttccgtccgcggcgaagtggtttcctccacctttgacgaaccggcgcaacgccacgtccaagttgccgaaatggtgcttgaaaaagccaagcgtatggtagaacacaaaaaagacgtggtcatcctgctggattcgattacccgccttgcccgcgcctacaataccgtcgtgcctacctcgggcaaaatcctaaccggcggtgtcgatgccaacgcactgcaccgccccaaacgtttcttcggcgcggcgcgcaacgtggaagaaggcggttcgctgaccatcatcgccaccgcattggttgaaaccggcagccgtatggacgatgtgatttacgaagaattcaaaggcaccggcaatatggaattgcaccttgaccgccgtatggcggaaaaacgcctcttccccgccatcaacatcaacaaatccggcacgcgccgcgaagagctgcttgtgccgaacgaccagttgcaacgtatgtggctcttacgcaagttcctgcacccgatggacgaaatcgaggcagccgaatttttaatcgggaaaatcaaagcctctaaaaacaatgatgatttctttgaactgatgcgcggcaaa

SEQ ID NO: 4 - protein, 419 aa, Neisseria meningitidis
MHVSELQTLHISKLLELAEEHGIENANRFRKQDLVFAIVRQMMKKGEGFTCSGTLEILPDGFGFLRSADTSYLAGPDDIYVSPTQIRRFNLHTGDTIEGSVRVPKDNERYFALVRLDTINGDHPEVCRHKILFENLTPLFPTEQLKLERDLKSEENLTGRAIDLISPIGKGQRALLVAPPKSGKTVMLQNIAHAVTSNYPEVELIVLLIDERPEEVTEMSRSVRGEVVSSTFDEPAQRHVQVAEMVLEKAKRMVEHKKDVVILLDSITRLARAYNTVVPTSGKILTGGVDANALHRPKRFFGAARNVEEGGSLTIIATALVETGSRMDDVIYEEFKGTGNMELHLDRRMAEKRLFPAININKSGTRREELLVPNDQLQRMWLLRKFLHPMDEIEAAEFLIGKIKASKNNDDFFELMRGK

SEQ ID NO: 5 - DNA, 599 bp, Neisseria meningitidis
  CDS (1)..(597)
atgaacaatttaatcaaaatcaacctcctcccctacagggaagagatgaacaagcgcaaacagcagcagtttaaaacgctgatgtacggtgccgtgctgacgggcgttgccgccgttgcggcaacctacctgtttatcgacaatatgatcaataaccagtcggaaagaaacacgctgctggaaacctccatcgcacacttggataccgagctgtcggaaatacaaaagctcaaacaggaaaaagatgccttcctgattaagaaaaacaaaatcgaggagctccagctcaaacgcctccaagccgcaaaaatcctcgacagcctgaatgaggccgtccccggaagcacctacctgacctcgctggatgccgttaccgccgactcttatcggctcagcggcaggacatccagcgacaaccgcgttgccgccatgatgagggcgatgcccaataccggcatattcaagcaacccgaattgttaagcatcaagaaaaacaattcgcatcaagaatttacccttcaggcaacattacaacccatcgtaaaggcggccgaatccaaagagaatccggcttcgggaaacgcacaggaggcaaactg

SEQ ID NO: 6 - protein, 199 aa, Neisseria meningitidis
MNNLIKINLLPYREEMNKRKQQQFKTLMYGAVLTGVAAVAATYLFIDNMINNQSERNTLLETSIAHLDTELSEIQKLKQEKDAFLIKKNKIEELQLKRLQAAKILDSLNEAVPGSTYLTSLDAVTADSYRLSGRTSSDNRVAAMMRAMPNTGIFKQPELLSIKKNNSHQEFTLQATLQPIVKAAESKENPASGNAQEAN

SEQ ID NO: 7 - DNA, 1773 bp, Neisseria meningitidis
  CDS (1)..(1773)
atgagtatcgtgctgcacggcgtggcggcgggcaaaggcattgccgtcggttgcgcccacctgattgcgcgcggtacggaggaagtgccgcagtatgatgttgcggaggcggacaccgatgccgaagccgaacgtttcgatgccgccgtcaaagccacgcgcaaagagttggaacagctccgcagcgcgattcccgaaaacgccccgaccgagttgggcgcgttcatctcgctgcacctgatgctcttgaccgatgttaccttgtcgcgcgaacccgtcgatattttaagggaacaaaaaatcaacgccgagtgggcattgaagcagcagagcgacaaactcgccgcccaattcgacaatatggacgatgcctatttgcgcgaacgcaagcaggatatgctgcaagtcgtccgccgcatccacaacaacctgatcgggcagggcaacgagttggaagttgccgacaacctgtttgacgaaaccgttctgattgcaaacgacctttcgcccgccgacacggttttgtttaaagagcagcgcattgccgccttcgttaccgatgccggcggccccaccgggcatacggcgattttgggcaggagcttggacatcccgtccgtcgtcgggctgcacaacgcgcgcaaactgattaccgagggcgaaacggtcattgtggacggtatcaacggcgtgttgattatcgcgccggatgagtcggtgttgaacgaataccgccgccgtgcccgcgaataccgcagccacaaacgcgatttgaacaagctcaaaaaaaccgccgccgccaccgctgacggggtctgcatcgagcttgtgggcaatatagaatccgccgaagacgtgaaaccgctgcacaacctcggcgcagacggcatcgggctgttccgcagcgagtttctttacctgaaccgcgatacgatgccgtctgaagacgagcagtacgaagtgtacagcgcgattgtcaaaaaaatgaaaggcaaaagcgtaacgatacggacagtcgatttgggtgtggacaaaaacccgcgctggttcgggaaaaacagcacgcccaacggcagcctcaaccccgcgctgggcatgaccggcatccgcctgtgccttgccgaaccggtcatgttccgcacccagatgcgcgccatcctccgcgccgccgcccacggccccgtgcggatgatgtggccgatgattacctccgtatccgaagtgcgccagtgcctcatccacctcgacaccgcgcaacgccagcttgccgaacgcggcgatgccttcggtaaagtcggcatcggctgtatgattgaaattccgtctgccgcgctgaccgtcggcagtattttgaaactggtcgatttcatctccgtcggtaccaacgacctgattcaatacatcttgtccgtcgatcgcggcgacgacagcgtcagccacctctaccagcccggccatcccgccgtgctgaaaatgctgcaacacgtcatccgtaccgccaaccgcatggacaaagacgtatccgtatgcggcgagatggcgggcgataccgcgtttacccgcgttttattgggtatggggctgcgccgtttttccatgaaccccaacaacatcctgcccgtcaaaaacatcattctgcacagcaatgtcggacagctcgaaagcgatattgtgaaagtcatccgctgcgaagacgaagagaagtcggaaaagctgatcaaacagatgaacagcgtgtctgtcgaggaagaagccgacttcaaggggcggaaa

SEQ ID NO: 8 - protein, 591 aa, Neisseria meningitidis
MSIVLHGVAAGKGIAVGCAHLIARGTEEVPQYDVAEADTDAEAERFDAAVKATRKELEQLRSAIPENAPTELGAFISLHLMLLTDVTLSREPVDILREQKINAEWALKQQSDKLAAQFDNMDDAYLRERKQDMLQVVRRIHNNLIGQGNELEVADNLFDETVLIANDLSPADTVLFKEQRIAAFVTDAGGPTGHTAILGRSLDIPSVVGLHNARKLITEGETVIVDGINGVLIIAPDESVLNEYRRRAREYRSHKRDLNKLKKTAAATADGVCIELVGNIESAEDVKPLHNLGADGIGLFRSEFLYLNRDTMPSEDEQYEVYSAIVKKMKGKSVTIRTVDLGVDKNPRWFGKNSTPNGSLNPALGMTGIRLCLAEPVMFRTQMRAILRAAAHGPVRMMWPMITSVSEVRQCLIHLDTAQRQLAERGDAFGKVGIGCMIEIPSAALTVGSILKLVDFISVGTNDLIQYILSVDRGDDSVSHLYQPGHPAVLKMLQHVIRTANRMDKDVSVCGEMAGDTAFTRVLLGMGLRRFSMNPNNILPVKNIILHSNVGQLESDIVKVIRCEDEEKSEKLIKQMNSVSVEEEADFKGRK

SEQ ID NO: 9 - DNA, 1572 bp, Neisseria meningitidis
  CDS (1)..(1572)
atgttcagacggtatcttccgaacagacagatgaatatggtttccaaactggacaaatactggcagcaccccgccctctactggcctttgctcatcctttttgccgccgccaccccctttaccttcgcaccctactaccacttttggctgatgcccttgattttcggtgccttcgtccgcctcatcgaactgcgtccgcgttttgctgtctcttccgcctacctgttcggcctgaccgcatacacgacacagttctactggatacacaccgccctgcacgacgtttccggcctgcccgacctctatgccgtaccgctgaccttcctactccccgcctaccttgccctttatccggcactgtgtttctggctgtggaaaaaatttaccctgcctcggggcataaaaatcggtttggtactgcccatcctgtggacactgaccgagtttgcccgcgaacgtttcctgaccggattcggctggggcgcaatcggctactcccaaatcaccccggacagcccgctcgccggctttgccccatttggcggcatccacatggttacactggcaaccgcctttctcggtgtctggctggttttggcgagtgataacaccgcacgttcgggcaaacgcctgcttccaattatcctgattgccgccctgcttgccgcaggctacaccgcccgacaaaccgacttcacccgccccgacggcagccgcagcaccgtcgccctgcttcaaggcaacatcgaccaaaccctcaaatggcgtgaagaccaagtcatcccgaccatacagaaatattacgaacaggtcggcaaaaccactgccgacatcgtcatcctgcccgaaaccgccatccccgtcatgcgccaaaacctgccggaaaacatactggcaaaatttgccgaacaggcgcaaaacaacggcagcgcgctcgccgtcggcatcagccaatacacttcggacggcaacggttacgaaaacgccgtcatcaacctgaccggttatcaggaaaacaatcaggacggcatcccctactacgccaaaaaccacctcgtccccttcggcgaatacaaaccgctgcccttcctgaccacgccgctttacaaaatgatgaatatgcccctttccgacttccgcaaaggcggcggtaagcaatccgccctgctgatgaaaaaccaaaaaatcgccttcaacatctgttacgaagacggattcggcgacgaactgattgccgccgccaaagatgccacactgcttgccaatgccagcaatatggcgtggtacggaaaatccaacgccatgtaccagcacctccaacaatcgcaggcgcgggctatggaactcggacgctatatggtccgtgccaccaacaccggcgcaaccgccatcatctcccccaaaggcaacatcatcgcccaagcccaacccgatacggaaaccgtattggaaggacacatcaaaggctatgtcggcgaaacgccctatatgaaaaccggcagttcatggtggttgatgggcatattgaccctagccgcactgattcttttcatcttccgaaacaaagaacac

SEQ ID NO: 10 - protein, 524 aa, Neisseria meningitidis
MFRRYLPNRQMNMVSKLDKYWQHPALYWPLLILFAAATPFTFAPYYHFWLMPLIFGAFVRLIELRPRFAVSSAYLFGLTAYTTQFYWIHTALHDVSGLPDLYAVPLTFLLPAYLALYPALCFWLWKKFTLPRGIKIGLVLPILWTLTEFARERFLTGFGWGAIGYSQITPDSPLAGFAPFGGIHMVTLATAFLGVWLVLASDNTARSGKRLLPIILIAALLAAGYTARQTDFTRPDGSRSTVALLQGNIDQTLKWREDQVIPTIQKYYEQVGKTTADIVILPETAIPVMRQNLPENILAKFAEQAQNNGSALAVGISQYTSDGNGYENAVINLTGYQENNQDGIPYYAKNHLVPFGEYKPLPFLTTPLYKMMNMPLSDFRKGGGKQSALLMKNQKIAFNICYEDGFGDELIAAAKDATLLANASNMAWYGKSNAMYQHLQQSQARAMELGRYMVRATNTGATAIISPKGNIIAQAQPDTETVLEGHIKGYVGETPYMKTGSSWWLMGILTLAALILFIFRNKEH

SEQ ID NO: 11 - DNA, 1185 bp, Neisseria meningitidis
  CDS (1)..(1185)
atgacagaggacgaacgtttcgcgtggctgcaattggcgtttacgccctatatcggcgcggaaagtttcctgctgctgatgcgccgtttcggcagcgcgcaaaatgccctgtccgcaccggcggaacaggtggcggcactgatacggcacaaacaggcgcttgaggcttggcgcaatgcggaaaaacgcgctctggcgcggcaggcggcagaagcggcgttggaatgggaaatgcgggacggatgccgcctgatgctgcttcaggatgaagatttccccgaaatgctgacgcaggggctgaccgcgccgccggttttgtttttgcgcggcaacgtgcggctgctgcacaagccttccgccgccatcgtcggcagccgccatgccacgccgcaggcgatgcggattgccaaagatttcggcaagtcgttgggcgggcaaaatatccccgttgtgtcgggtatggcttccggtatcgatactgccgcccatcagggcgcactggaggcagaaggcggcaccatcgccgtgtgggggacgggtatagaccgcatttacccgccgtccaataaaaaccttgcctatgaaatcgccgaaaaaggattgattgtcagcgagtttcccatcggtacgcgcccgtatgccggcaattttccgcgccgcaaccgcctgattgccgccctgtcgcaagtaacgctggtggttgaagccgccttggaatccggttcgctgattaccgccggcctggcggcagaaatggggcgcgaagtgatggcggtacccggttcgatagacaatccgcacagcaaaggctgccacaaactgattaaagacggcgcaaaattggtggaatgcctggacgacatcctgaacgaatgcccggggctattgcaaaatacgggtgcttcatcatattctataaataaggatacgcccgacacgggacgccgcgccgttcagacggcatacgccccgccgcctgccgcaaaaatgccgtctgaagcggcggcaggcggcacagccgtcggcggcatactggataaaatgggtttcgaccccatccatcccgacgtgcttgccggacagttggctatgcctgccgcagatttgtatgccgcactgttggaattggaattggacggcagcgttgccgcaatgcccagcggcagataccagcgtatccgaact

SEQ ID NO: 12 - protein, 395 aa, Neisseria meningitidis
MTEDERFAWLQLAFTPYIGAESFLLLMRRFGSAQNALSAPAEQVAALIRHKQALEAWRNAEKRALARQAAEAALEWEMRDGCRLMLLQDEDFPEMLTQGLTAPPVLFLRGNVRLLHKPSAAIVGSRHATPQAMRIAKDFGKSLGGQNIPVVSGMASGIDTAAHQGALEAEGGTIAVWGTGIDRIYPPSNKNLAYEIAEKGLIVSEFPIGTRPYAGNFPRRNRLIAALSQVTLVVEAALESGSLITAGLAAEMGREVMAVPGSIDNPHSKGCHKLIKDGAKLVECLDDILNECPGLLQNTGASSYSINKDTPDTGRRAVQTAYAPPPAAKMPSEAAAGGTAVGGILDKMGFDPIHPDVLAGQLAMPAADLYAALLELELDGSVAAMPSGRYQRIRT

SEQ ID NO: 13 - DNA, 804 bp, Neisseria meningitidis
  CDS (1)..(804)
atgccgacagttttcatctttttgacggcggtttacgcaggattgggtgcatttgcatggcactgccaacagcaggggctcggccgggattacccgtggaagacggaattgccggttttgggcgcggcattgaccgtccatggcgcggcactgcttatgccggtcattcaagacaaaatcatcattatgggctttgggtattccggcagcctgattgtttggatgatgctgtttatttattttgccggcagcttcttttattcgctgcgcggagtgcagttgctgctgtatccttgcgccgcactgatgctgctgtcaggtttggtttttcctggaaaattctcgggatatgaaattaccgaccttccctttatgctgcatatcggaacttcgctgcttgcatacgggctgttcggcatcgcaacattattgtccgttttgaccctgctgctgaatcggagcctgcaccgcaggaacttctccaagctcgcaggattcctgccgtcgctgctcagtttggaaaaactcatgttccaggccatgtgggcaggtttcatcctgctgacctattccgtcgtcagcggaacattttttgccgaagccgtattcggcaaacccatgacctttacccataaaaccgtattcggcatattgtcatggctgatttacggcggactgctgctcaagcacagcatgaccgcatggcgcggcaaaaaagccgccgtgtggaccatcatcggatttgtcagcctgatgattgcctatatgggcagcaagttcgtattggaaatcattctgaacaga

SEQ ID NO: 14 - protein, 268 aa, Neisseria meningitidis
MPTVFIFLTAVYAGLGAFAWHCQQQGLGRDYPWKTELPVLGAALTVHGAALLMPVIQDKIIIMGFGYSGSLIVWMMLFIYFAGSFFYSLRGVQLLLYPCAALMLLSGLVFPGKFSGYEITDLPFMLHIGTSLLAYGLFGIATLLSVLTLLLNRSLHRRNFSKLAGFLPSLLSLEKLMFQAMWAGFILLTYSVVSGTFFAEAVFGKPMTFTHKTVFGILSWLIYGGLLLKHSMTAWRGKKAAVWTIIGFVSLMIAYMGSKFVLEIILNR

SEQ ID NO: 15 - DNA, 2391 bp, Neisseria meningitidis
  CDS (1)..(2391)
atgtcaacagtccaaatgaataaaaatattaaatctttaaatttacgggaaaaagacccgtttttaagtcgtgaaaaacagcgttatgaacatcctttgcccagtcgggaatggataatcgagctgcttgaacgtaaaggcgtaccatccaagattgaagctttggtacgcgaattgtcgattaaggaagaagagtacgaatttttcgaacgtcgtctgaaggcgatggcgcgggacggtcaggttttaatcaaccgtcggggcgcggtttgcgcggcggacaaattggatttggtcaaatgccgtgtcgaggcgcacaaagacggcttcggtttcgccgtgccgctcacgcccgccaaagacggtgattttgttttgtacgaacgccagatgcgcggcattatgcacggcgatattgtcactgttcgtcctgccggcatggaccgtaggggccgccgcgaagggacggttctggatattgtcgaacgcgcgcaaagcaaagtggtcggccgtttctatatggataggggcgtggcgattttggagccggaagacaagcgtctgaaccaaagcatcgtattggaaccggacggcgtggcgcgtttcaaacctgaatccggtcaggtcatcgtcggcgaaattgaggtttatcctgagcaaaaccggccggcagtggcaaaaatcatcgaagttttgggcgattatgccgacagcggcatggagattgaaattgccgtgcgcaagcatcatttgccgcaccaattcagtgaagcgtgtgccaaagccgcgaaaaaaattcccgaccatgtacgcaaaagcgatttgaaaggccgcgtcgatttgcgcgacctgcctttggtaacgatagacggcgaaacggctcgagattttgacgatgcggtgtttgccgagaaaatcggacgcaattaccgtctggtcgtggcgattgccgatgtcagccattatgtccgccccgatgacgctatcgacacggacgctcaggaacgcagcaccagtgtttacttcccgcgccgcgtgattcccatgttgccggaaaacctgtccaacggcatctgctcgctcaatcctcatgtcgagcgtttgtgtgtggtgtgcgatatggttatcacttacgcgggcaatatcaaagaataccgcttctaccccgccgtgatgcgctctcatgcccgcctgacctacaaccaagtttggaaatggctttcaggcggcatcgagcatccgttcaaaacccaaatcgacacgctttacaaactcttcaaaatccttcagaaaaagcgtttcgaacgcggggcggtggagtttgacagcatcgaaacccaaatgcttttcgacgacaacggtaaaattgaaaaaatcgtccccgttgtccgcaacgatgcccacaagctgattgaagaatgtatgttggcggcaaacgtttgcgcagcggattttctgttgaaaaacaagcataccgcattgttccgcaaccatttggggcccacgcccgaaaaactcgccgccttgcgcgagcagctcggtctgttggggcttcaacttggcggcggcgacaacccgtcgccgaaagactatgccgcgcttgccggacagttcaaaggcaggccggatgccgaattgctgcaagtcatgatgttgcgctccatgcaacaggcggtttacgaaccgcattgcgacggacactttggtcttgcctacgaagcatacgcccacttcacctcgcccatccgccgctatcccgacctgaccgtacaccgcgccatcaaagccgtgttgaatcagcaaacctacacgccaaaaaaaagctggcaggctttgggcgtgcatacctcgttctgtgagcgccgtgccgacgacgccagccgcgacgtggaaaactggctgaaaacctattatatgcgcgataaggtcggcgaagtattcgaaggtaaaatctccggcatgaccagttttggtatctttgtaacactggacggcatccacattgacggcttggtgcatatcagcgatttgggcgaagactatttcaacttccgccccgaaatcatggcaatcgaaggcgaacgcagcggcatccgtttcaacatgggggacagggttgccgtccgggtcgcccgtgccgatttggatgacggaaaaatcgattttgtcctgattgccggggggagcggcagggggcggaaagttaaatcatccgcgtctgccaaaccggcagggacggcggggaaagggaagccgaaaaccgccgccgagaaaaaaacagcccgaggcggcaaagtaaggggaaggggcgcgtctgccgccgcagaatcgaggaaaaaggcaaagaaaccggttccgattaaggtaaaaaaacggaaaggcaaatcataa

SEQ ID NO: 16 - protein, 796 aa, Neisseria meningitidis
MSTVQMNKNIKSLNLREKDPFLSREKQRYEHPLPSREWIIELLERKGVPSKIEALVRELSIKEEEYEFFERRLKAMARDGQVLINRRGAVCAADKLDLVKCRVEAHKDGFGFAVPLTPAKDGDFVLYERQMRGIMHGDIVTVRPAGMDRRGRREGTVLDIVERAQSKVVGRFYMDRGVAILEPEDKRLNQSIVLEPDGVARFKPESGQVIVGEIEVYPEQNRPAVAKIIEVLGDYADSGMEIEIAVRKHHLPHQFSEACAKAAKKIPDHVRKSDLKGRVDLRDLPLVTIDGETARDFDDAVFAEKIGRNYRLVVAIADVSHYVRPDDAIDTDAQERSTSVYFPRRVIPMLPENLSNGICSLNPHVERLCVVCDMVITYAGNIKEYRFYPAVMRSHARLTYNQVWKWLSGGIEHPFKTQIDTLYKLFKILQKKRFERGAVEFDSIETQMLFDDNGKIEKIVPVVRNDAHKLIEECMLAANVCAADFLLKNKHTALFRNHLGPTPEKLAALREQLGLLGLQLGGGDNPSPKDYAALAGQFKGRPDAELLQVMMLRSMQQAVYEPHCDGHFGLAYEAYAHFTSPIRRYPDLTVHRAIKAVLNQQTYTPKKSWQALGVHTSFCERRADDASRDVENWLKTYYMRDKVGEVFEGKISGMTSFGIFVTLDGIHIDGLVHISDLGEDYFNFRPEIMAIEGERSGIRFNMGDRVAVRVARADLDDGKIDFVLIAGGSGRGRKVKSSASAKPAGTAGKGKPKTAAEKKTARGGKVRGRGASAAAESRKKAKKPVPIKVKKRKGKS

SEQ ID NO: 17 - DNA, 252 bp, Neisseria meningitidis
  CDS (1)..(252)
atggagcagtcgggcaaattcagttggtctgcggcagctttttgggacattccctaccccgccaccaggcggattgcctcaagtttgtattcgaccgaatattttgtcgtatgctttctacgtttgatgccactctctccgtgtaatctgtattttgtcacccatctgcgtaccaatgaatcggaaatagaaagatggtctgctgttccctgccaaatagtattgaacgacggcaagtcggaattcatctga

SEQ ID NO: 18 - protein, 83 aa, Neisseria meningitidis
MEQSGKFSWSAAAFWDIPYPATRRIASSLYSTEYFVVCFLRLMPLSPCNLYFVTHLRTNESEIERWSAVPCQIVLNDGKSEFI

SEQ ID NO: 19 - DNA, 789 bp, Neisseria meningitidis
  CDS (1)..(789)
atgaatgtttattttctcggcggcggcaatatggcggctgccgttgcgggcggattggtcaaacaaggcggttaccgcatccatatagccaatcggggtgcggaaaaacgcgaacgtttgggaaaagagttgggggtcgaaacttcggcaaccctgccggagcttcattccgacgatgttttaatccttgccgtcaaaccgcaggatatggaagccgcgtgcaaaaatatccgcaccaatggtgcattggtgctttctgtcgcagctggattgtcggttgatacgctcagccgttacctcgggggaacacgccgcattgtccgggttatgccgaatacacccggaaaaatcgggctgggcgtatccggtatgtttgccgaagcggaagtatcggaaacagaccgcaggattgccgatcgaatcatgaaatcagtcggtttgaccgtttggttggaagatgaagcgcaaatgcacagcattaccggtatcagcggcagcgggccggcatatgtgttttatctgctggacgcattgcaaaatgccgccatccgacaagggtttgatatggcagaagcacgcgcgctcagtctggcaacgtttaaaggagcggttgcccttgccgagcagacgggtgaagatttcgagaagcttcaaaaaaatgtaacgtcaaaaggcgggacaacccacgaagccgtggaagctttcaggcggcatcgtgtcgccgaagccataagcgagggcgtttgtgcctgtgtgcgccgttcgcaggaaatggaacggcaatatcaa

SEQ ID NO: 20 - protein, 263 aa, Neisseria meningitidis
MNVYFLGGGNMAAAVAGGLVKQGGYRIHIANRGAEKRERLGKELGVETSATLPELHSDDVLILAVKPQDMEAACKNIRTNGALVLSVAAGLSVDTLSRYLGGTRRIVRVMPNTPGKIGLGVSGMFAEAEVSETDRRIADRIMKSVGLTVWLEDEAQMHSITGISGSGPAYVFYLLDALQNAAIRQGFDMAEARALSLATFKGAVALAEQTGEDFEKLQKNVTSKGGTTHEAVEAFRRHRVAEAISEGVCACVRRSQEMERQYQ

SEQ ID NO: 21 - DNA, 132 bp, Neisseria meningitidis
  CDS (1)..(132)
atggggaaacggctgatgccgtctgaagggaaaggtgcggcaggcaatccggaggcgcataaaaaagcagatatattcggactgcacctcccgaatatatctgcctgctgtttcctctttattcagccttta

SEQ ID NO: 22 - protein, 44 aa, Neisseria meningitidis
MGKRLMPSEGKGAAGNPEAHKKADIFGLHLPNISACCFLFIQPL

(19) United States
(12) Patent Application Publication
(10) Pub. No.: US 2003/0170261 A1
(43) Pub. Date: Sep. 11, 2003

(54) Virulence genes, proteins, and their use

(76) Inventor: Christoph Tang, London (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/276,629

(22) PCT Filed: May 18, 2001
(86) PCT No.: PCT/GB01/02247

(30) Foreign Application Priority Data
May 18, 2000 (GB) 0012079.0

(51) Int. Cl. A61K 39/02

(52) U.S. Cl. 424/190.1

(57) ABSTRACT

A series of genes form Neisseria meningitidis are shown to encode products which are responsible for DNA uptake. The identification of these genes therefore allows attenuated microorganisms to be produced that have a reduced ability to take up DNA. Microorganisms of the invention may be used in the production of genetically stable mutant microorganisms. The genes or their encoded products can be used in the manufacture of vaccines for therapeutic application.

### FIELD OF THE INVENTION

This invention relates to bacterial genes and proteins, and their use. More particularly, it relates to genes and proteins/peptides obtained from Neisseria meningitidis, and their use in therapy and in screening for drugs.

### BACKGROUND OF THE INVENTION

Neisseria meningitidis is a Gram-negative bacterial pathogen that is implicated in septic shock and bacterial meningitis. This bacterium is a leading cause of bacterial meningitis in developed countries, and causes large-scale epidemics in Africa and China. In the UK, Neisseria meningitidis is the leading cause of death in childhood apart from road traffic accidents. The bacterium naturally inhabits the human naso-pharynx and then gains access to the blood stream from where it causes severe septicaemia or meningitis. Although current anti-microbials are effective in eliminating the bacterium from the body, the mortalilty from menigococcal septicaemia remains substantial.

Immunisation with avirulent N. meningitidis microorganisms has also been proposed. Avirulence is achieved through genetic modification to disrupt the expression of genes implicated in virulence. One concern is that a mutant strain may revert to its virulent form by taking up replacement DNA. It is therefore desirable to provide means for treating or preventing conditions caused by Neisseria meningitidis, e.g. by immunisation.

### SUMMARY OF THE INVENTION

The present invention is based on the discovery of genes in Neisseria meningitidis that are required for natural competence (uptake and incorporation of exogenous DNA).

The genes identified herein may be utilised to prepare microorganisms that are transformation-deficient. The mutant microorganisms will usually have a mutation that disrupts the expression of one or more of the genes identified herein, to provide a strain that lacks the ability to uptake DNA. These microorganisms will also have use in therapy and diagnosis.

According to a first aspect of the invention, a microorganism comprises a mutation that disrupts the expression of a gene comprising any of the nucleotide sequences identified in claim 1, or a homologue thereof with at least 40% sequence identity, for therapeutic or diagnostic use.

The microorganisms may have many therapeutic uses for treating Neisseria infections, including use in vaccines for prophylactic application.

According to a second aspect, a peptide is encoded by an operon including any of the nucleotide sequences identified in claim 1.

### DESCRIPTION OF THE INVENTION

The present invention is based on the discovery of genes encoding peptides which are responsible for the uptake of DNA. The peptides and genes of the invention are therefore useful for the preparation of therapeutic agents to treat infection. It should be understood that references to therapy also include preventative treatments, e.g. vaccination. Furthermore, while the products of the invention are intended primarily for treatment of infections in human patients, veterinary applications are also considered to be within the scope of the invention.

The present invention is described with reference to Neisseria meningitidis. However, all the Neisseria strains, and many other Gram-negative bacterial strains are likely to include related peptides or proteins having amino acid sequence identity or similarity to those identified herein. Organisms likely to contain the peptides include, but are not limited to the genera Salmonella, Enterobacter, Klebsiella, Shigella and Yersinia.

Preferably, the peptides that may be useful in the various aspects of the invention have greater than a 40% similarity with the peptides identified herein. More preferably, the peptides have greater than 60% sequence similarity. Most preferably, the peptides have greater than 80% sequence similarity, e.g. 95% similarity. With regard to the polynucleotide sequences identified herein, related polynucleotides that may be useful in the various aspects of the invention may have greater than 40% identity with the sequences identified herein. More preferably, the polynucleotide sequences have greater than 60% sequence identity. Most preferably, the polynucleotide sequences have greater than 80% sequence identity, e.g. 95% identity.

The terms “similarity” and “identity” are known in the art. The use of the term “identity” refers to a sequence comparison based on identical matches between correspondingly identical positions in the sequences being compared. The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

Levels of identity between gene sequences and levels of identity or similarity between amino acid sequences can be calculated using known methods. In relation to the present invention, publicly available computer based methods for determining identity and similarity include the BLASTP, BLASTN and FASTA (Atschul et al., J. Molec. Biol., 1990; 215:403-410), the BLASTX program available from NCBI, and the Gap program from Genetics Computer Group, Madison Wis. The levels of similarity and identity provided herein, were obtained using the Gap program, with a Gap penalty of 12 and a Gap length penalty of 4 for determining the amino acid sequence comparisons, and a Gap penalty of 50 and a Gap length penalty of 3 for the polynucleotide sequence comparisons.

Having characterised a gene according to the invention, it is possible to use the gene sequence to search for related genes or peptides in other microorganisms. This may be carried out by searching in existing databases, e.g. EMBL or GeneBank.

Peptides or proteins according to the invention may be purified and isolated by methods known in the art. In particular, having identified the gene sequence, it will be possible to use recombinant techniques to express the genes in a suitable host. Active fragments and related molecules can be identified and may be useful in therapy. For example, the peptides or their active fragments may be used as antigenic determinants in a vaccine, to elicit an immune response. They may also be used in the preparation of antibodies, for passive immunisation, or diagnostic applications. Suitable antibodies include monoclonal antibodies, or fragments thereof, including single chain Fv fragments. Methods for the preparation of antibodies will be apparent to those skilled in the art.

Active fragments of the peptides are those that retain the biological function of the peptide, i.e. retain the ability to take up DNA. Typically, the fragment will be at least 30 nucleotides (10 amino acids) in size, preferably 60 nucleotides (20 amino acids) and most preferably greater than 90 nucleotides (30 amino acids) in size.

The genes identified herein are likely to be useful both in generating vaccine strains that cannot take up exogenous DNA, and as a target for antimicrobials. Attenuated microorganisms can be produced with an attenuating mutation in a virulence gene or genes together with a mutation that disrupts a gene or genes of the invention, providing an attenuated microorganism that cannot take up extracellular DNA.

The term “virulence” is known in the art and refers to the pathogenicity of a microorganism. The use of the term “avirulent” is intended therefore to refer to microorganisms that are no longer pathogenic.

Suitable virulence genes will be apparent to the skilled person, and include auxotroph genes and those identified in Sun et al., Nature medicine, 2000; 6(11): 1269-1273.

The skilled person will be aware of methods for disrupting the expression of particular genes. Techniques that may be used include insertional inactivation or gene deletion techniques. Attenuated microorganisms according to the invention may also comprise additional mutations in other genes.

The preparation of vaccines based on attenuated microorganisms is known to those skilled in the art. Vaccine compositions can be formulated with suitable carriers or adjuvants, e.g. alum, as necessary or desired, to provide effective immunisation against infection. The preparation of vaccine formulations will be apparent to the skilled person.

Attenuated microorganisms may also be used as carrier systems for the delivery of heterologous antigens, therapeutic proteins or nucleic acids (DNA or RNA). In this embodiment, the attenuated microorganisms are used to deliver a heterologous antigen, protein or nucleic acid to a particular site in vivo. Introduction of a heterologous antigen, peptide or nucleic acid into an attenuated microorganism can be carried out by conventional techniques, including the use of recombinant constructs, e.g. vectors, which comprise polynucleotides that express the heterologous antigen or therapeutic protein, and also include suitable promoter sequences. As the microorganisms are transformation-deficient, it will be necessary to incorporate heterologous nucleic acids via an active mechanism, e.g. electroporation. This is a known technique. Alternatively, the gene that encodes the heterologous antigen or protein may be incorporated into the genome of the organism and the endogenous promoters used to control expression. A further alternative is to modify the microorganisms to disrupt the genes identified herein after first transforming with the heterologous gene.

More generally, and as is well known to those skilled in the art, a suitable amount of an active component of the invention can be selected, for therapeutic use, as can suitable carriers or excipients, and routes of administration. These factors would be chosen or determined according to known criteria such as the nature/severity of the condition to be treated, the type and/or health of the subject etc.

The various products of the invention may also be used in veterinary applications.

The following is a brief overview of the experimental procedure used to identify the genes.

Signature-tagged mutagenesis (STM) (Hensel et al., Science, 1995; 269: 400-403) was used to identify the genes in N. meningitidis that are essential for uptake of DNA. In STM, individual mutants are tagged with unique sequence identifiers, allowing large numbers of mutants to be analysed simultaneously.

A Neisseria meningitidis serogroup B Tn10 mutant bank was used to screen for mutants that exhibited a transformation defect. Pools of mutants were transformed with either plasmid or chromosomal DNA carrying an erythromycin-resistance marker. Transformation-deficient mutants were identified by plating out transformed pools of mutant bacteria onto solid media, in the presence and absence of erythromycin and performing a STM screen. Bacteria containing a modified version of the transposon Tn10 inserted within a competence gene failed to be recovered from plates containing erythromycin inoculated with a mixed population of mutants, and were, therefore likely to be defective in the uptake of exogenous DNA.

The cloned Neisseria meningitidis nucleotide sequences immediately following the Tn10 insertion were identified, and their translated sequences characterised. Homologues were then searched for in existing protein and DNA databases (www.sanger.ac.uk/Projects/N-meningitidis; www.tigr.org; and www.ncbi.nlm.nih.gov).

The selected mutants were tested for the ability to take up exogenous DNA and were shown to take up exogenous DNA at levels at least 5 fold below wild-type levels. As the genes are transcribed as part of an operon it is possible that the mutation is due to a polar effect on a downstream gene.

To prevent the identification of mutants that merely could not express an erythromycin resistant or capsule-minus phenotype, all putatively competence-defective mutants were also analysed for their ability to be transformed to tetracycline resistance. The results confirmed that the mutants were transformation deficient.

The genes of the invention are shown in Table 1 together with the identification of any known homologue. In addition, Table 1 shows the transformation frequency, illustrating the deficiency of DNA uptake exhibited by the mutants, compared to wild-type.

TABLE 1

| SEQ | | | Transformation | Accession |
|---|---|---|---|---|
| ID NO. | Genes | Function | frequency | number |
| 1 | recG | ATP-dependent DNA helicase RECG | 1/5 | NMB1788 |
| 3 | rho | Transcription termination factor RHO | 0 | NMB0617 |
| 5 | pilN | PBP1 | 0 | NMB1809 |
| 7 | phlI | Phosphoenolpyruvate-protein | 1/30 | NMB2044 |
| | | phosphotransferase | | |
| 9 | cutE | E. coli apolipoprotein N-acyltransferase | 1/10 | NMB0713 |
| 11 | smf | SMF protein (DNA processing chain A) | 0 | NMB0116 |
| 13 | | CcsA-related protein | 1/2000 | NMB0296 |
| 15 | vacB | E. coli Vacb protein | 1/10 | NMB1200 |
| 17 | | No hit in NM nucleic acid database | 1/5 | |
| 19 | proC | Pyrroline-5-carboxylate reductase | 0 | NMB055 |
| 21 | | Phorphoribosylglycinamide | 1/40 | NMB1566 |
| | | formyltransferase | | |

1. A microorganism comprising a mutation that disrupts the expression of a gene comprising any of the nucleotide sequences identified herein as SEQ ID NOS. 1, 3, 5, 7, 9, 11, 13, 15, 17, 19 and 21, or a related molecule having at least 40% sequence identity, for therapeutic use.

2. A microorganism according to claim 1, wherein the mutation results in insertional inactivation or a gene deletion.

3. A microorganism according to claim 1 or claim 2, wherein the microorganism is Neisseria meningitidis.

4. A microorganism according to any preceding claim, further comprising a mutation that disrupts the expression of a virulence gene.

5. A microorganism according to any preceding claim, comprising a heterologous antigen, therapeutic peptide or nucleic acid.

6. A microorganism according to any preceding claim, wherein the sequence identity is at least 60%.

7. A microorganism according to any preceding claim, wherein the sequence identity is at least 90%.

8. A microorganism according to any of claims 1 to 5, wherein the gene normally encodes a peptide comprising any of the amino acid sequences identified herein as SEQ ID NOS. 2, 4, 6, 8, 10, 12, 14, 16, 18, 20 or 22.

9. A vaccine comprising a microorganism according to any preceding claim.

10. A peptide encoded by an operon including any of the nucleotide sequences identified herein as SEQ ID NOS. 1, 3, 5, 7, 9. 11, 13, 15, 17, 19 and 21 of N. meningitidis, or a related molecule having at least 40% sequence similarity or identity at the peptide or nucleotide level in a Gram-negative bacterium, or a functional fragment thereof, for therapeutic or diagnostic use.

11. A peptide according to claim 10, wherein the sequence similarity or identity is at least 60%.

12. A peptide according to claim 10 or claim 11, wherein the sequence similarity or identity is at least 90%.

13. A peptide according to claim 10, comprising the amino acid sequence identified herein as SEQ ID NOS. 2, 4, 6, 8, 10, 12, 14, 16, 18, 20 and 22.

14. A polynucleotide encoding a peptide according to any of claims 10 to 14, or its complement, for therapeutic or diagnostic use.

15. An antibody raised against a peptide according to any of claims 10 to 13.

16. Use of a product according to any of claims 1 to 14, for the manufacture of a medicament for use in the treatment or prevention of a condition associated with infection by Neisseria or Gram-negative bacteria.

17. Use according to claim 16, wherein the condition is meningitis.

18. Use according to claim 16 or claim 17, for veterinary treatment.
